(19) United States
(12) Patent Application Publication
(10) Pub. No.: US 2024/0400621 A1
(43) Pub. Date: Dec. 5, 2024

(54) PEPTIDES FOR METABOLIC SYNDROME

(71) Applicant: ALPARIS, S.A. de C.V., Mexico City (MX)

(72) Inventors: Juan Pablo SENOSIAIN PELÁEZ, Mexico City (MX); Karim Enrique JAÉN CHÁVEZ, Chihuahua (MX)

(21) Appl. No.: 18/691,280

(22) PCT Filed: Sep. 12, 2022
(86) PCT No.: PCT/IB2022/058593

§ 371 Date: Mar. 12, 2024

(30) Foreign Application Priority Data
Sep. 13, 2021 (MX) MX/A/2021/011153

(51) Int. Cl. C07K 14/195; A61K 38/00; A61P 3/10

(52) U.S. Cl. CPC C07K 14/195; A61P 3/10; A61K 38/00

(57) ABSTRACT

The present invention concerns peptides derived from SEQ ID NO:1 with in vitro and in vivo therapeutic activity that have advantageous properties for their industrial production and medicinal formulation. The present invention also refers to the use of such peptides for the treatment and prevention of diseases, specifically, metabolic disorders, hyperglycemia, insulinemia, hypercholesterolemia, and/or hypertension; they are also useful for modulating immune, metabolic, and endocrine signaling, as well as intestinal barrier function, and maintaining the homeostasis of glucose, insulin, adiponectin, leptin, interleukins, cholesterol, and triglycerides; in addition, their formulation in pharmaceutical compositions that comprise pharmaceutically acceptable excipients which can be administered orally, parenterally, topically, sublingually, respiratorily, transdermally, or any combination thereof.

### FIELD OF THE INVENTION

The present invention belongs to the field of therapeutic peptide engineering, for the treatment of conditions or diseases via the modulation of the Toll-like receptor, TLR 2 and TLR4.

### BACKGROUNDS OF THE INVENTION

AMUC_1100 is a membrane protein of the Gram-negative bacterium Akkermansia muciniphila that has been linked to immune homeostasis of the intestinal mucosa, an improved intestinal barrier function, and has been noted as a therapeutic candidate for the treatment of metabolic disorders.

The state of the art describes various therapeutic applications for AMUC_1100. For example, it is used for the treatment of inflammatory bowel disease in WO2020226438A1; for the treatment of metabolic disorders, including obesity and diabetes, in CN107903310A; to improve emotional memory in CN111690044A; to treat diseases in the oral cavity in US20200397831A1; as an immunotherapeutic treatment of malignant tumors in CN110559424A; and, more generally, to modulate immune signaling and intestinal barrier functioning, and to maintain glucose, cholesterol, and triglyceride homeostasis in WO2016177797A1.

Nonetheless, there is not yet an AMUC_1100-based pharmaceutical product with proven therapeutic efficacy. So far there is no information on the three-dimensional structure of AMUC_1100; according to the National Center for Biotechnology Information (NCBI), the primary structure of AMUC_1100 consists of 317 amino acid residues. As a starting point, for the expression of the peptides of the present invention, this sequence was optimized by removing the signal peptide sequence and adding an Xa factor and histidine tag to facilitate their identification, as shown in SEQ ID NO:1.

AMUC_1100 is a relatively large protein, as well as hydrophobic, unstable, and with poor solubility. This poses difficulties in its large-scale manufacturing, as well as in its formulation. Some efforts have focused on modifying the AMUC_1100 protein in order to improve its properties. CN110950937A discloses a peptide that corresponds to the first 60-90 amino acids of the amino terminal of AMUC_1100, and peptides that correspond to the first 90 amino acids of the amino terminal of this protein, in which at least one hydrophobic amino acid is mutated (mutations L68R, L72E, Y75E, A76R, V79E, and X85E). US20180311310A1 obtains fragments of AMUC_1100 and identifies peptide motifs related to TLR2 binding. However, none of these documents affords peptides with proven therapeutic efficacy or that are suitable for the manufacture of medicines.

Consequently, the need to contribute therapeutic molecules that are effective for the treatment of diseases and that have suitable characteristics for production and medicinal formulation remains.

### BRIEF DESCRIPTION OF THE DRAWINGS

FIG. 1. Generation of a polyclonal antibody ANTI SEQ ID NO:1

FIG. 2. Prediction of receptor-binding sites and disordered regions of SEQ ID NO: 1.

FIG. 3. Verification of the expression of SEQ ID NO:3 and SEQ ID NO:4 by electrophoresis in agarose gel. Lane 1: Molecular Weight Marker, Lane 2: Expression of SEQ ID NO: 4 (no induction), Lane 3: Expression of SEQ ID NO: 4 (induced), Lane 4: Expression of SEQ ID NO: 3 (no Induction), Lane 5: Expression of SEQ ID NO: 3 (induced), Lane 6: Expression of SEQ ID NO: 1.

FIG. 4. RP-UPLC chromatogram of the purified fraction SEQ ID NO: 3 at 214 nm. The arrow indicates the peak of SEQ ID NO: 3

FIG. 5. Mass spectrum of SEQ ID NO:3. Peak deconvolution with retention time of 96.3 min using HPLC MS/MS. The most abundant fraction has a mass of 16140.26 Da.

FIG. 6. Chromatograms of the separation of the purified fraction SEQ ID NO: 4 by RP-UPLC at 214 and 280 nm. The arrow indicates the peak of SEQ ID NO: 4

FIG. 7. Mass spectrum of SEN ID NO: 4. Peak deconvolution with retention time of 105 min using HPLC MS/MS. The most abundant fraction has a mass of 17524 Da.

FIG. 8. Peptide mapping of proteolysis of SEQ ID NO: 3 with trypsin. The numbers identify the peptides according to Table 3.

FIG. 9. Peptide mapping of proteolysis of SEQ ID NO: 3 with Lys-C. The numbers identify the peptides in accordance with Table 4.

FIG. 10. Stimulation of hTLR2 with different concentrations of the peptide SEQ ID NO: 2.

FIG. 11. Dose-response of SEQ ID NO: 2. The points correspond to the mean, and the error bars to the standard deviation. The line is the curve fitt of the 4-parameter sigmoidal model.

FIG. 12. Stimulation of hTLR2 with different concentrations of the peptide SEQ ID NO: 4.

FIG. 13. Dose-response relationship of SEQ ID NO: 4. The points correspond to the mean, and the error bars to the standard deviation. The line is the curve fit of the 4-parameter sigmoidal model.

FIG. 14. Stimulation of hTLR2 with different concentrations of the peptide SEQ ID NO: 3.

FIG. 15. Dose-response relationship of SEQ ID NO: 3. The points correspond to the mean, and the error bars to the standard deviation. The line is the curve fit of the 4-parameter sigmoidal model.

FIG. 16. Stimulation of hTLR2 with a proteolyzed peptide of SEQ ID NO: 3.

FIG. 17. Stimulation of hTLR2 with fractions of the purified peptides of the proteolysate of SEQ ID NO:3.

FIG. 18. Stimulation of hTLR2 with fractions of peptides of the proteolysate of SEQ ID NO: 3 purified with ultrafiltration.

FIGS. 19A and 19B. Area beneath the curve of the glucose tolerance of mice at 0 weeks and 12 weeks treated with DN, DAG, DAG+F2, high dose of DAG+F3, and low dose of DAG+F3, **** means the difference was significant with a p-value<0.0001; *** means the difference was significant with a p-value<0.001; ** means the difference was significant with a p-value<0.01; and * means the difference was significant with a p-value<0.05.

FIGS. 20A and 20B. Area beneath the insulin resistance curve of mice at 0 weeks and 12 weeks treated with DN, DAG, DAG+F2, high dose of DAG+F3, and low dose of DAG+F3, **** means the difference was significant with a p-value<0.0001; *** means the difference was significant with a p-value<0.001; ** means the difference was significant with a p-value<0.01; and * means the difference was significant with a p-value<0.05.

FIG. 21. Insulin levels in mice fed DN, DAG, DAG+F2, high dose of DAG+F3 (0.6 μg), and low dose of DAG+F3 (0.2 μg) at 12 weeks. ** means the difference was significant with a p-value<0.01; and * means the difference was significant with a p-value<0.05.

FIG. 22. IL-β levels in mice fed DN, DAG, DAG+F2, high dose of DAG+F3, and low dose of DAG+F3 at 12 weeks. **** means the difference was significant with a p-value<0.0001; *** means the difference was significant with a p-value<0.001; ** means the difference was significant with a p-value<0.01; and * means the difference was significant with a p-value<0.05.

FIG. 23. TNFα levels in mice fed DN, DAG, DAG+F2, high dose of DAG+F3, and low dose of DAG+F3 at 12 weeks. **** means the difference was significant with a p-value<0.0001; *** means the difference was significant with a p-value<0.001; ** means the difference was significant with a p-value<0.01; and * means the difference was significant with a p-value<0.05.

FIG. 24. IL-6 levels in mice fed DN, DAG, DAG+F2, high dose of DAG+F3 (0.6 μg), and low dose of DAG+F3 (0.2 μg) at 12 weeks. **** means the difference was significant with a p-value<0.0001; *** means the difference was significant with a p-value<0.001; ** means the difference was significant with a p-value<0.01; and * means the difference was significant with a p-value<0.05.

FIG. 25. Leptin levels in mice fed DN, DAG, DAG+F2, high dose of DAG+F3 (0.6 μg), and low dose of DAG+F3 (0.2 μg) at 12 weeks. **** means the difference was significant with a p-value<0.0001.

FIG. 26. Adiponectin levels in mice fed DN, DAG, DAG+F2, high dose of DAG+F3 (0.6 μg), and low dose of DAG+F3 (0.2 μg) at 12 weeks. ** means the difference was significant with a p-value<0.01; and * means the difference was significant with a p-value<0.05.

FIG. 27. Design of the construction of the optimized sequence for the expression of the gene that encodes SEQ ID NO. 1.

### SUMMARY OF THE INVENTION

The present invention refers to peptides derived from SEQ ID NO: 1 with therapeutic activity in vitro and in vivo, and which has advantageous properties for its industrial production and medicinal formulation. The present invention also relates to the use of these peptides for the treatment and prevention of diseases.

In particular, the present invention refers to peptides that consist of amino acid sequences that have at least 60% sequence identity with any of the sequences in SEQ ID NO: 2, SEQ ID NO: 3, and SEQ ID NO: 4.

SEQ ID NO: 1 is an outer membrane protein located in the bacterium Akkermansia muciniphila. This protein has been investigated because of its relationship with the energetic metabolism of the host. In particular, the state of the art has linked Akkermansia muciniphila to body weight, fat, and blood glucose levels, as well as the regulation of the host subject's immune system. The protein SEQ ID NO:1 is believed to contribute to this activity.

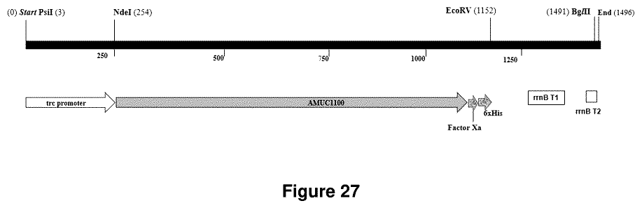

A synthetic gene was designed that encodes the protein SEQ ID NO. 1 with the general characteristics of the molecular structure (a strong and inducible promoter, a histidine tag, a stop codon, transcription terminators, and restriction sites). FIG. 27.

The resulting sequence has a size of 864 bp. In addition, the sequence was optimized based on the preferential use of E. coli codons using GenScript's OptimumGene program.

The peptides of the present invention may be full-length protein fragments of SEQ ID NO: 1, where the fragments have a length of no more than 180 amino acids. In certain aspects, the fragments have no more than 168 amino acids, no more than 160 amino acids, no more than 155 amino acids, no more than 150 amino acids, no more than 145 amino acids, no more than 143 amino acids, no more than 140 amino acids, no more than 130 amino acids, no more than 120 amino acids, no more than 110 amino acids, no more than 100 amino acids, no more than 90 amino acids, no more than 80 amino acids, no more than 70 amino acids, no more than 60 amino acids, no more than 50 amino acids, no more than 45 amino acids, no more than 40 amino acids, no more than 35 amino acids, or no more than 32 amino acids.

The peptides of the invention can have a minimum size of 30 amino acids, such as 31 amino acids, 32 amino acids, 33 amino acids, 34 amino acids, 35 amino acids, 37 amino acids, 40 amino acids, 45 amino acids, 50 amino acids, 100 amino acids, 110 amino acids, 112 amino acids, 120 amino acids, 130 amino acids, 135 amino acids, 139 amino acids, 140 amino acids, 145 amino acids, 149 amino acids, 150 amino acids, 160 amino acids, 170 amino acids, or 180 amino acids.

Consequently, the size of the peptides of the invention may vary between the minimum and maximum described above. For example, in certain aspects, the peptide of the invention has a length between 30 and 45 amino acids, or a length between 112 and 168 amino acids, or a length between 120 and 180 amino acids.

In one aspect, the peptides of the present invention consist of an amino acid sequence that is at least 60% identical to any of the sequences corresponding to SEQ ID NO: 2, SEQ ID NO: 3, and SEQ ID NO: 4.

In optional aspects of the application, the peptides have a percentage of at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with respect to any of the sequences in SEQ ID NO: 2, SEQ ID NO: 3, and SEQ ID NO: 4.

Preferably, the peptides have a percentage identity of at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with respect to any of the sequences in SEQ ID NO: 2, SEQ ID NO: 3, and SEQ ID NO: 4.

The present invention also refers to a peptide comprising an amino acid sequence that has at least 80% sequence identity with any of the sequences in SEQ ID NO: 2, SEQ ID NO: 3, and SEQ ID NO: 4, where the peptide has no more than 280 amino acids in length, for example, no more than 250 amino acids, no more than 230 amino acids, no more than 200 amino acids, no more than 180 amino acids, no more than 160, no more than 140, no more than 120, no more than 100 amino acids, no more than 90 amino acids, no more than 80 amino acids, no more than 70 amino acids, no more than 60 amino acids, or no more than 50 amino acids. In this case, the peptides of the invention can be considered as included or contained within a peptide of longer length, and preferably, the activity of the peptides of the invention is preserved. As described, the amino acid sequence preferably has more than 80% sequence identity with SEQ ID NO: 2, SEQ ID NO: 3, and SEQ ID NO: 4, such as at least 83%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity.

The numbering of the amino acid sequence is done by counting from the N-terminal end (also called the amino terminal or N-terminal).

The “percentage of identity” with respect to peptides, polypeptides, or proteins, as referred to in this document, has the meaning commonly attributed by the state of the art, and as such refers to the percentage of amino acids that are identical between two amino acid sequences, which are compared after an optimal alignment of these sequences, and where this percentage is merely statistical and the differences between the two amino acid sequences are randomly distributed along the sequence. “Optimal alignment” is the alignment of amino acid sequences that results in a higher percentage of identity. The percentage of identity is calculated by determining the number of identical positions in which an amino acid is identical in the two compared sequences, dividing the number of identical positions by the number of compared positions, and multiplying the result by 100 to obtain the percentage of identity between the two sequences. The sequence comparison between two amino acid sequences can be performed manually or by using computer programs known in the state of the art, such as the BLAST (Basic Local Alignment Search Tool) algorithm.

Preferably, the peptides of the invention have a defined amino acid sequence of either SEQ ID NO: 2, SEQ ID NO: 3, and SEQ ID NO: 4.

Optionally, when the peptides of the invention have some variation with respect to the amino acid sequences defined in SEQ ID NO: 2, SEQ ID NO: 3, and SEQ ID NO: 4, such sequence variants retain the activity of the reference peptide. Accordingly, the present invention also refers to sequence variants that have 1, 2, 3, 4, 5, 6, 7, 8, or 10 substituted, eliminated, and/or added amino acids, preferably without nullifying the peptide activity of SEQ ID NO: 2, SEQ ID NO: 3, or SEQ ID NO: 4.

The peptides of the present invention can be obtained by protein production methods that are known in the state of the art.

In some aspects, the full-length protein of SEQ ID NO: 1 can be fragmented by chemical or enzymatic means, cutting the protein until the peptides of the present invention are obtained. For example, in the case of enzymatic cutting, calpain-1, caspase-1, trypsin, or Lys-C can be used, or any other enzyme that is capable of cutting at the desired sites. The selection of suitable enzymes to obtain the peptides of the invention from a longer peptide should be apparent to a technician in the subject matter.

It is also possible to chemically synthesize the peptides of the present invention. Biological synthesis is another alternative, using expression systems, preferably bacterial, that are suitable for the production of peptides, polypeptides, or proteins. The chemical and biological synthesis methods available and suitable for manufacturing the peptides of the invention should be apparent to a technician in the subject matter.

The present invention also relates to a nucleic acid molecule that encodes a peptide according to the present invention. Preferably, the nucleic acid molecule is selected from the group consisting of SEQ ID NO: 5, SEQ ID NO: 6, and SEQ ID NO: 7.

In other aspects, the invention refers to an expression vector comprising a nucleic acid molecule operationally attached to a promoter, as well as a host cell comprising one or more of these expression vectors.

Thus, another aspect of the invention refers to a method of producing any of the peptides of the invention, which involves culturing the host cell of the invention under conditions that cause the expression of the peptide and separating the peptide from the culture. Preferably, the peptide of the invention is secreted by the host cell and isolated from the culture medium. In another modality, the invention also refers to the production, separation, extraction, and/or purification of any of the peptides of the invention.

The peptides of the invention are particularly useful because of their reduced size and physicochemical properties. Compared to the original protein, the peptides of the invention are at least 50% smaller, which in principle facilitates their large-scale production and enables their chemical synthesis. In addition, the peptides of the present invention have higher solubility and stability than that of the AMUC_1100 protein. Consequently, the peptides of the present invention have the advantage of being more easily incorporated into a wide variety of formulations. Advantageously, the peptides of the present invention are also more easily absorbed in the organism due to their reduced size, physicochemical properties, stability, and solubility.

It should be noted that there is a high uncertainty that functional fragments of AMUC_1100 exist. To date, no binding sites of the protein have been identified, and although bioinformatic predictions could be made about the possible binding sites, this does not guarantee that there is activity in the fragments that contain them. Additionally, the number of obtainable fragments from the complete protein is so large that the identification of active fragments would be an insurmountable task for a technician in the subject matter. Despite the above, the inventors found that the peptides of the present invention not only show in vitro activity, but also proved effective in preclinical studies in mice, confirming the high clinical potential of the invention.

The present invention also relates to a composition comprising a peptide of the invention as described. In preferred aspects, the composition comprises any of the peptides of SEQ ID NO: 2, SEQ ID NO: 3, and SEQ ID NO: 4. Such composition, among others, can be nutraceutical or pharmaceutical. In the case of a pharmaceutical composition, said composition includes a pharmaceutically acceptable carrier.

Within the scope of this invention, a pharmaceutical carrier is a material, composition, or vehicle involved in carrying or transporting an active ingredient, and which, in accordance with a reasonable medical judgment, is acceptable to administrate into the human or animal body without an undesirable complication or response, with a reasonable risk-benefit ratio. Examples of pharmaceutically acceptable excipients include diluents, solubilizers, emulsifiers, binders, preservatives, and/or adjuvants.

The pharmaceutical composition of the present invention may be formulated for administration via any suitable route, including oral, sublingual, buccal, topical, transdermal, or parenteral.

The peptides of the invention are useful in the treatment of diseases. Therefore, the compounds of the invention can be used in the treatment of inflammatory diseases, chronic diseases, metabolic and endocrine disorders, metabolic syndrome, obesity, diabetes, weight loss/control, decrease in body fat, intestinal disorders, insulinemia, hyperglycemia, hypertension, and inflammatory bowel disease; in addition, they are useful for modulating immune signaling and the function of the intestinal barrier, and maintaining glucose, cholesterol, and triglyceride homeostasis, or as an immunotherapeutic treatment of malignant tumors.

Accordingly, the present invention also refers to a method of treating a disease, disorder or condition that involves administering a therapeutically effective amount of the peptide of the invention to a patient. The type of disease, disorder, or condition to be treated with the peptides of the invention will be apparent to a technician in the subject matter, based on the present disclosure.

Another embodiment of the present invention is the treatment and prevention of diseases, specifically, metabolic disorders, hyperglycemia, insulinemia, hypercholesterolemia, and/or hypertension with the peptides of the present invention with the peptides of the present invention.

Another embodiment of the present invention is the modulation of immunological, metabolic, endocrine signaling and intestinal barrier function, and maintaining homeostasis of glucose, insulin, adiponectin, leptin, interleukins, cholesterol and triglycerides.

Another embodiment is the peptides use to prepare drugs for the treatment and prevention of inflammatory, metabolic and endocrine disorders, hyperglycemia, insulinemia, hypercholesterolemia, hypertension, modulating immune signaling, intestinal barrier function, maintaining glucose homeostasis, insulin, adiponectin, leptin, interleukin, cholesterol and/or triglycerides.

Another embodiment is the peptides use in the treatment and prevention of inflammatory, metabolic and endocrine disorders, hyperglycemia, insulinemia, hypercholesterolemia, hypertension, modulating immune signaling, intestinal barrier function, maintaining glucose homeostasis, insulin, adiponectin, leptin, interleukin, cholesterol and/or triglycerides.

### DESCRIPTION OF THE EMBODIMENTS

### Expression and Selection of Clones for the Production of SEQ ID NO. 1

For the expression and selection of clones for the production of SEQ ID NO. 1 in E. coli, the constructs AMUC1100S_pET26b (+) and AMUC1100_COTROLjs_pET26b (+) were designed for the expression of SEQ ID NO. 1 in the E. coli strains W3110 and BL21 (DE3), based on the defined protein sequence. These constructs were synthesized by GenScript USA (Piscataway, NJ). The manufacturer's quality control documentation, as well as genetic testing performed in our laboratory, indicate that the 6 kb plasmids, AMUC1100S_pET26b (+) and AMUC1100_CONTROL_pET26b (+), contain the sequence and genetic elements originally contemplated for the recombinant expression of SEQ ID NO. 1 in E. coli. The plasmids were used to transform the E. coli strains. SDS-PAGE analysis of the cultures of the transformed strains revealed that SEQ ID NO. 1 was expressed in most of the clones of E. coli, W3110 with AMUC1100S_pET26b (+), and E. coli BL21 (DE3) with AMUC1100_CONTROL_pET26b (+), with a band of the expected molecular weight, 32 kDa. The identity of SEQ ID NO. 1 was corroborated via Western blot immunodetection with an anti-tag histidine antibody.

After a selection process based on the expression of SEQ ID NO. 1 in culture with an LBfitone medium, 1 mM IPTG, and 30° C., the best clone of E. coli W3110 AMUC1100S_PET26b (+) had a specific yield of 63±7 mg/gPCH, while the best clone of E. coli BL21 (DE3) AMUC1100_CONTROL_pET26b (+) had a specific yield of 38±2 mg/gPCH. In addition, SEQ ID NO:1 was found mainly in the soluble fractions in both expression systems.

Overexpression of SEQ ID NO. 1 in E. coli was confirmed using molecular biology and immunodetection techniques. The best producing clones were selected from both strains and expression systems, and an initial evaluation was made of the best induction conditions. The W3110 AMUC1100S_pET26 b (+) system was found to produce the highest amount of SEQ ID NO. 1, with 230 mg/L. This clone was cultivated for the production and purification of SEQ ID NO. 1 in the later stages of the invention.

### Production of SEQ ID NO. 1

The production of SEQ ID NO. 1 by the E. coli W3110 and E. coli BL21 (DE3) strains was evaluated using different concentrations of IPTG and lactose, with W3.110 being the best producing strain of SEQ ID NO. 1. With 1 mM IPTG, it had yields of 27.4 mg/gpCH and 100 mg/L; while with 30 mM lactose, it was 29.4 mg/gpcH and 141 mg/L. BL21 (DE3) production with 1 mM IPTG had yields of 13.9 mg/gpci-i and 55 mg/L; while with 15 mM lactose, it was 2.8 mg/gpcH and 17.5 mg/L. Although lactose was effective in W3110 and has important advantages over the use of IPTG, high variability in the yields was observed. Because of this, induction with 0.5 mM IPTG was chosen, which was the condition that resulted in high and reproducible yields. For scale-up, the W3110 strain was cultured in 2 L of LB-phytone medium in an instrumented bioreactor, which produced 335 mg/L of SEQ ID NO. 1.

Regarding the production of SEQ ID NO. 1, the volumetric and specific yields were 4.8 and 1.7 times higher, respectively, than those observed in flask. This may be due to the higher rate of O₂ transfer in the bioreactor and pH control compared to cultures in flask, as both O₂ limitation and an increase in pH negatively impact cell growth and recombinant protein production (Shang et al. 2009).

### Purification of SEQ ID NO. 1

The protein of SEQ ID NO. 1, expressed in recombinant form in this invention, contains a tag of six residues of histidine at the carboxyl terminal end, allowing it to be purified by immobilized metal cation affinity chromatography (IMAC). Purification was initially evaluated with the soluble fractions of the two strains that were used for recombinant production, obtained by enzymatic lysis and sonication. The first approach with the Ni-NTA Agarose resin allowed for the recovery of 6% of the SEQ ID NO. 1 with a purity of 50-80%. An FPLC Ákta Prime chromatographic system and an XK16/20 column packed with 13 mL of Chelating Sepharose Fast Flow-Ni⁺² resin were used for purification. A minimum imidazole concentration of 30 mM was defined in both the sample to be injected and the equilibrium buffer. Elution was performed with 500 mM imidazole at 6.5 mL/min, resulting in a minimum recovery of 80% of SEQ ID No. 1 with 82% purity. The ultrafiltration of the tangential flow of SEQ ID NO. 1 during the chromatography stage, in order to concentrate and change the buffer, negatively impacted protein quality. However, the dialysis of EQ ID NO. 1 in the PBS buffer or 0.9% NaCl did not affect its quality.

The absence of protein in the permeate demonstrated the good retention of the UF membrane, in which the total recovery of the protein was achieved. However, the aggregation of SEQ ID NO. 1 during buffer change was a first indicator that this process had an impact on the stability of the protein. This was corroborated by the SDSPAGE analysis, in which the remaining protein in the soluble fraction presented fragments of lower molecular weight than that of SEQ ID NO. 1, but greater than 25 kDa. On the other hand, the precipitate turned out to be denatured SEQ ID NO. 1, but not degraded.

Damage to proteins in this type of operation is common and is attributable to factors such as the cutting force, turbulence, and the interaction between phases, which is why surfactants such as polysorbate 20 and 80 were used, which confer protection against protein denaturation (Callahan et al., 2014). Shortening the buffer change time by using a membrane with a larger filtration surface, implementing temperature control of the entire system, and minimizing the exposure of the gas-liquid phases are some measures that can mitigate this problem. However, the fact that the protein is not stable in the PBS pH 7.4 buffer cannot be excluded.

The anion, cation, and IMAC exchange operations were not effective in recovering the undegraded and soluble SEQ ID NO. 1. They did, however, provide information about the nature of the degraded fragments. All of these fragments were negatively charged at pH 7.4 and retained the histidine tag (in whole or in part). Separation by IMAC further revealed that the SEQ ID NO. 1 can be fully recovered from 150 mM imidazole.

### Method and Evaluation of the Biological Activity of SEQ ID NO. 1

The analysis of the biological activity of the protein of SEQ ID NO. 1 was performed via an in vitro Toll-like receptors (TLRs) recognition assay. The HEK-Blue hTLR2 cell line expressing the hTLR2 receptor was used, whose stimulation activates the NF-Kβ factor, which in turn induces the production of the reporter protein, secreted embryonic alkaline phosphatase (SEAP). The SEAP levels are quantified by a colorimetric method with the reagent QUANTI-Blue and correspond to hTLR2 stimulation levels. The HEK-Blue hTLR2 cell line was thawed and propagated to make a master cell bank. From this bank, a vial was thawed to check the correct growth of the cell line and the absence of mycoplasma contamination. The cells of the thawed vial were propagated to make a working cell bank and continue the subcultures to mount the activity assay of SEQ ID NO. 1. This consisted of a series of tests in which the cellular response was evaluated with different molecules to determine the most suitable conditions for the assay.

With 24 h of stimulation of hTLR2 and 60 min of incubation at 37° C. of the reaction of QUANTI-Blue with the cell culture supernatants, it was found that the SEQ ID PROTEIN NO. 1 (10 μg/mL) expressed in E. coli bound to hTLR2, with a relative activation of NF-κβ of 16.6%±2.7% (with respect to the culture medium), which is comparable to what was observed by Plovier et al. (2017). The determined EC50 for SEQ ID NO. 1 was 1147±211 ng/ml (35.1±6.4 nM).

### Bioinformatic Analysis of SEQ ID NO. 1

Ordered and disordered regions, as well as potential receptor binding sites, were predicted from the peptide sequence of SEQ ID NO. 1. From this analysis, it was found that the protein has a highly ordered, alkaline, and serine-abundant domain at its amino-terminal end, which was designated SEQ ID NO. 4. A potential receptor binding site was located at the carboxyl terminal end. This domain is inherently disordered, acidic, and rich in prolines, and was designated SEQ ID NO. 3.

Alignment of the peptide sequences of SEQ ID NO. 1, SEQ ID NO. 4, and SEQ ID NO. 3 with the AMUC_1100 sequence claimed in application EP15166598.1 resulted in homologies of about 90%, 46%, and 43%, respectively. The 10% difference in the complete protein is due to the absence of a signal peptide in SEQ ID NO. 1 produced in the laboratory. Predictions of the proteolytic cleavage sites in SEQ ID NO. 1 were analyzed, and it was found that caspase-1 can generate fragments similar to SEQ ID NO. 4 and SEQ ID NO. 3. Additionally, peptides that can be generated from proteolysis with trypsin and Lys-C were obtained, which can serve as reference for the proteomic analysis of these proteins and the search for the minimum biologically active region. SEQ ID NO. 1, SEQ ID NO. 4, and SEQ ID NO. 3 were considered to be unstable and hydrophobic, but soluble.

### Identification of SEQ ID NO: 3 and SEQ ID NO: 4 by Bioinformatic Analysis

A comprehensive bioinformatics analysis of SEQ ID NO:1 was performed using the bioinformatics tools, PSIPRED and DISOPRED3. Surprisingly, two regions with different characteristics were identified; in FIG. 2, the region near the terminal carboxyl (from amino acid 151 to 300) comprises an intrinsically disordered domain formed mostly by twists, acidic and rich in prolines, and was designated SEQ ID NO: 3, in which some potential receptor binding sites were also located. Meanwhile, the end of the amino terminal end of SEQ ID NO: 1 (from amino acid 1 to 150) was distinguished by a highly ordered domain comprised mostly of beta sheets and alpha helices, alkaline and abundant in serine, which was designated SEQ ID NO: 4.

Another aspect of the prediction is that most of the rigid structural motifs are concentrated in the amino terminal end SEQ ID NO: 4, while the flexible structural motifs are concentrated in the carboxyl terminal end SEQ ID NO: 3.

FIG. 2 clearly shows the 4 disordered regions between the amino acids 1-20, 150-170, 220-250 and 290-300, in which 3 receptor-binding domains were predicted on the amino acids 1-12, 154-168, and 284-300. The prediction of binding sites at positions 1-12 and 284-300 reinforces the validity of the bioinformatic analysis, given that these positions correspond to the sequence preceded by the signal peptide in the native version of the protein and the histidine tail tag.

### Example 3. Purification of Peptides SEQ ID NO: 3 and SEQ ID NO: 4

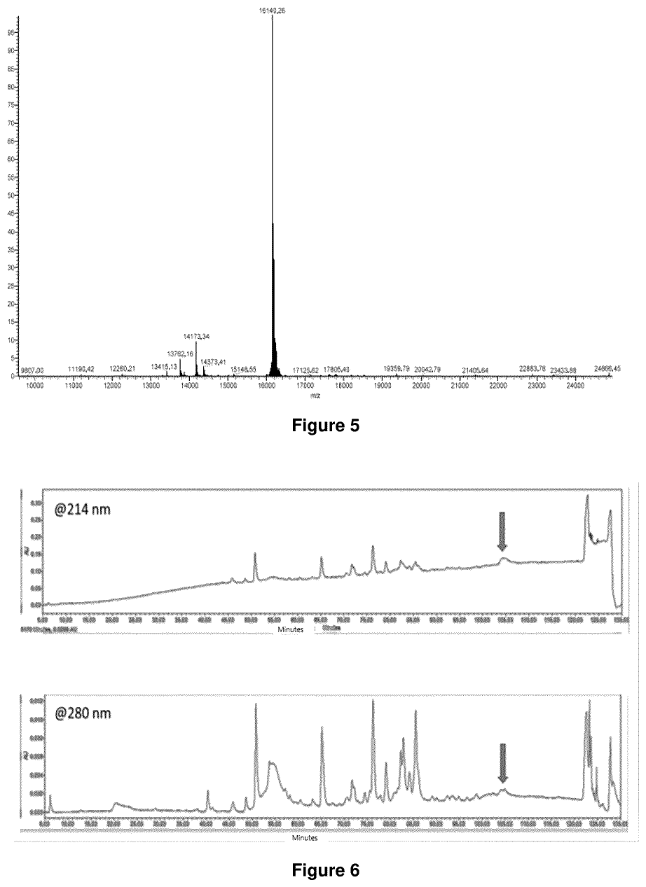

SEQ ID NO: 3 was found in the soluble fraction of lysis, and purification was performed in 2 steps, first by immobilized metal cation affinity chromatography using a Chelating Sepharose Fast Flow resin, and second by anion exchange using a Fractogel TMAE HiCap (M) resin. The purification of SEQ ID NO:3 was also achieved in a single step by anion exchange using a Ni-NTA Fast Flow resin. Finally, SEQ ID NO: 3 was subjected to a 5 kDa ultrafiltration analysis on a Spin-X unit. The purity of SEQ ID NO:3 was >90%. The purified fraction of SEQ ID NO: 3 was analyzed via ultra-performance liquid chromatography (UPLC); FIG. 4 shows the chromatogram at 214 nm, where the blue arrow indicates the peak that corresponds to SEQ ID NO: 3. After the scanning and deconvolution of this peak, an experimental mass of 16147 kDa was defined for SEQ ID NO: 3, very close to the theoretical PM of 16141 kDa (FIG. 5).

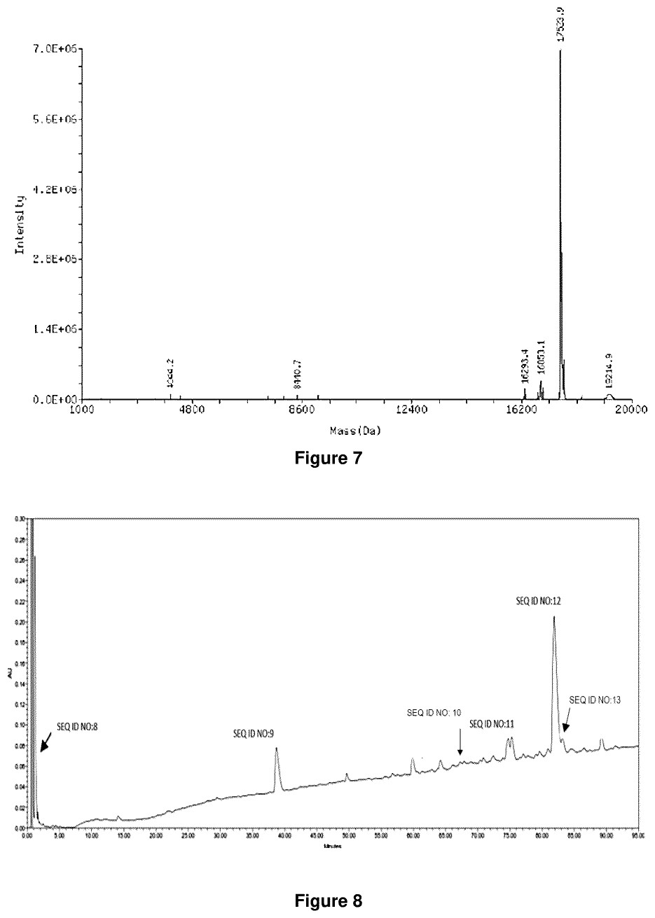

SEQ ID NO: 4 was found mainly in the insoluble fractions of lysis. To recover the insoluble fraction, the soluble fraction was discarded and the insoluble fraction was enriched by proteases, then solubilized with the help of an extraction buffer. The purification was performed by immobilized metal cation affinity chromatography using a Ni-NTA Agarose resin. The purified fraction of SEQ ID NO:4 was analyzed by ultra-performance liquid chromatography (UPLC). FIG. 6 shows the chromatograms at 214 and 280 nm. After scanning and deconvolution, the peak had a mass of 17524 Da (FIG. 7) with a retention time of 105 min, very close to the expected theoretical mass of 17517 Da. The purity of SEQ ID NO: 4 was >80%.

### Recovery and Purification of Peptide SEQ ID No. 4

Enrichment of the insoluble fraction. In order to maximize the recovery and purity of the peptide of SEQ ID NO. 4, the insoluble fraction was dissolved in 10 ml of Bugbuster Master Mix. The suspension was incubated at RT for 30 min, stirring every 10 min for 30 s. The suspension was centrifuged at 12,000 g for 10 min at 10° C. The soluble fraction was discarded.

Solubilization of the insoluble fraction. The enriched insoluble fraction was dissolved in 5 ml of extraction buffer. 1 mL aliquots of the resulting solution were prepared in 1.5 mL tubes and centrifuged at 14,000 rpm for 10 min at 10° C. The supernatant was recovered and filtered using 0.22 μm syringe filters. The resulting solution was diluted with an equivalent volume of base buffer.

Purification by IMAC using Ni-NTA Agarose resin. A 5 mL gravity-flow column was packed with 1 mL of Ni-NTA Agarose resin. This was balanced with a VC equilibrium buffer, and then the load was passed. The column was flushed with 10 VC of equilibrium buffer and eluted with 5 VC of elution buffer. The unbound, washed, and eluted fractions were collected.

The eluted fraction was dialyzed in 2 L of 20 mM acetate buffer for 24 h in refrigeration. The solution of SEQ ID NO. 4 was recovered and aseptically filtered with a 0.22 μm syringe filter in a laminar flow hood for refrigerated storage.

With this procedure, the soluble SEQ ID NO. 4 was obtained with a yield of 0.6 mg/gBPH and a purity of 81.4%, determined by DS-PAGE. SEQ ID NO. 4 shows a PM of 17524 Da.

### Recovery and Purification of Peptide of SEQ ID No. 3

1.5 ml of elution buffer and 2.5 ml of 5 M NaCl were added to 21 ml of the recovered soluble fraction. The resulting solution was filtered with a 0.22 μm syringe filter.

Purification by IMAC using Chelating Sepharose Fast Flow resin. An Akta Pime Plus chromatographic system and an XK-16 column packed with 13 ml of Chelating Sepharose Fast Flow resin were used for purification, placing the equilibrium buffer in port A1 and the elution buffer in port B. The chromatographic run was performed at a flow rate of 6.5 ml/min. The column was balanced with 10 VC of equilibrium buffer, and the load was injected through port A2. After rebalancing the column with 5 VC of equilibrium buffer, it was washed with 5 VC of 75 mM imidazole (15% B) and subsequently eluted with 5 VC of 500 mM imidazole (100% B). The eluted fraction began to collect when the A280 nm was greater than 0.05 mAU and was terminated when it was less than 0.11 mAU.

Al purification using Fractogel TMAE HiCap (M) resin. To increase purity, a second purification step was performed using the Akta Prime Plus chromatographic system and a column pre-packed with 5 ml of Fractogel EMD TMAE HiCap (M) resin, placing the equilibrium buffer in the A1 port and the elution buffer in port B. The chromatographic run was performed at a flow rate of 2.5 ml/min. The column was washed with 10 VC of equilibrium buffer, washed with 5 VC of 100 mM NaCl (10% B), and eluted with 5 VC of 250 mM NaCl (25%).

The eluted fraction was dialyzed in 2 L of PBS buffer using a 32 mm Spectra/Por membrane with a cutoff of 6-8 kDa for 24 h in refrigeration. The solution containing SEQ ID NO. 3 was recovered and aseptically filtered with a 0.22 μm syringe filter in a laminar flow hood for refrigerated storage.

SEQ ID NO. 3 has a mass of 16140 Da, along with 2 proteolyzed forms at its amino terminal end with masses of 14178 Da and 13767 Da.

Up to 20 mg of SEQ ID NO. 3 can be recovered from the lysis of a 5.4 gBPH cell pellet, i.e., a yield of 3.7 mg F2/gBPH.

Finally, SEQ ID NO. 4 and SEQ ID NO. 3 showed no visible aggregation in the ultrafiltration operations, so they can be concentrated and diafiltered in systems with PES membranes of 5 kDa cutoff, such as Spin-X units or Viva Flow cassettes in a TFF unit.

### Evaluation of Biological Activity of Peptides Obtained by Proteolysis of SEQ ID NO. 1

SEQ ID NO. 3 is a peptide derived from SEQ ID NO. 1 with 16.1 kDa, which has an EC50 similar to that of SEQ ID NO. 1, but lower than that of the peptide SEQ ID NO. 4 on activation of the hTLR2 receptor. To find the minimal region in SEQ ID NO. 1 capable of activating hTLR2, peptides were generated under native conditions via enzymatic proteolysis of SEQ ID NO. 3 with the proteases trypsin and Lys-C Sequencing Grade. By assaying the biological activity of SEQ ID NO. 3 at a concentration of 305 nM, it was found that trypsin treatment resulted in peptides that did not significantly stimulate hTLR2, whereas treatment with Lys-C resulted in peptides that stimulated hTLR2 at the same level as SEQ ID NO. 3.

The proteolyzed peptide of SEQ ID NO. 3 with Lys-C stimulates hTLR2, and its biological activity is greater than that of the proteolyzed peptide of SEQ ID NO. 3 with trypsin. The peptides SEQ ID NO. 12, SEQ ID NO. 14, and SEQ ID NO. 16 do not stimulate hTLR2.

### Production of Anti-SEQ ID NO. 1 Polyclonal Antibodies

In order to have reagents for the identification and detection of SEQ ID NO: 1, two rabbits were immunized for the production of polyclonal antibodies, which were purified and characterized. The anti-SEQ ID NO. 1 polyclonal antibodies present in the rabbit serum had titers in the order of 1.07×10⁴. The sera from both immunized rabbits detected up to 125 ng of SEQ ID NO. 1, specifically using a 1:10,000 dilution.

The antibodies in the sera were purified by affinity chromatography with and without fractional precipitation, with a recovery efficiency of 67% and 94%, respectively. Both were obtained with a purity of more than 90%. The purified antibodies were put in sterile storage at −80° C.; half of them (30-50 ml) with cryoprotectant and antimicrobial agents, and the other half without additives. These antibodies were able to detect up to 20 ng of the protein of SEQ ID NO. 1 using a 1:10,000 dilution. The volume obtained from the purified antibodies is sufficient to perform up to 2,225,000 ELISA or Western blot assays.

Two female rabbits with no more than 3 months of age, identified as “56” and “57”, were used for production. The rabbits were immunized 3 times every 3 weeks by injecting a 1 mL dose with 500 μg of SEQ ID NO. 1 subcutaneously into the dorsal part. The first 2 immunization doses contained AIFs, which were prepared by mixing 200 μL of SEQ ID NO. 1 in PBS pH 7.4 (Lot 186-52A; REP-SNOI-04) with 300 pL PBS and 500 pL AIF (Lot KJ135408). The third dose was formulated with PBS only; the AIF was eliminated.

An ELISA assay based on the SOP-ANA-320 protocol was used to determine the anti-SEQ ID NO. 1 antibody titers. 100 μL of a 5 μg/mL solution of SEQ ID NO. 1 were added to a high-adhesion 96-well plate, with a sensitization buffer (pH 9.5) in each well, and then incubated for 16 h at 2-8° C. The sensitized plate was washed 5 times with 200 μL of a wash solution, and 125 μL of blocking solution was added to each well for blockage, then incubated at 37° C. for 2 h. The blocked plate was washed 5 times with a wash solution, then dilutions of the sera were added to column 1, described as follows: 1:5, preimmune and first bleeding; 1:50 bleeding; 1:100, third bleeding; all in a final volume of 150 pL per well with a reaction buffer. 1:3 serial dilutions were mixed, taking 50 pL from the wells of the first column and mixing them with 100 μL of the reaction buffer from the wells of the second column and so on, until 10 serial dilutions were obtained. The plate was incubated at 37° C. for 2 h. After incubation, the plate was washed 5 times with 200 μL of wash solution, and 100 μL of a 1:1500 dilution of the anti-rabbit secondary antibodies, coupled with alkaline phosphatase in reaction buffer, was added. The plate was incubated at 37° C. for 1 h. Finally, the washes were repeated, and 100 μL of a PNPP solution at 120 μg/mL was added to develop the plate. After 10 min of incubation at room temperature and observing coloration in the wells, 100 μL of 2M NaOH was added to stop the reaction. Lastly, the absorbance was measured at 405 nm on the microplate reader.

The antibodies from the serum were mainly accompanied by albumin and transferrin (Grodzki & Berenstein, 2010, Antibody Purification: Ammonium Sulfate Fractionation or Gel Filtration. In: Oliver C., Jamur M. (eds) Immunocytochemical Methods and Protocols. Methods in Molecular Biology (Methods and Protocols), vol 588. Humana Press). These contaminants can interfere with detection processes (Huse et al., 2002, Purification of antibodies by affinity chromatography. Journal of Biophysical Methods. 51:217-231), so the need arises to eliminate them through processes that commonly involve fractional precipitation and chromatography, either with ion exchange or affinity, in which typically 70% of IgG with a purity of 95% is recovered (Chon and Zarbis-Papastoitsis, 2011, Advances in the production and downstream processing of antibodies. New Biotechnology. 28 (5): 458-463). The addition of a cold-saturated solution of ammonium sulfate is the most commonly used method to precipitate the antibodies from the serum, which in addition to concentrating them, decreases the load volume and impurity content for subsequent operations. It has been observed that 80-95% of IgG from rabbit typically precipitates 33% to 40% of the ammonium sulfate saturation (Hebert et al, 1973. Determination of the Optimal Ammonium Sulfate Concentration from the Fractionation of Rabbit, Sheep, Horse, and Goat Antisera. Applied Microbiology. 25 (1): 26-36); Morissette et al, 1990, Production of Avid Rabbit Antibodies Against Staphylococcal Enterotoxins A and B. Journal of Food Protection. 53 (9): 782-785; Grodzki and Berenstein, 2010; Mariam et al., 2015, Purification of rabbit polyclonal immunoglobulin G with ammonium sulphate precipitation and mixed-mode chromatography. Separation and Purification Technology. 144:133-138). On the other hand, affinity chromatography with protein A, a Staphylococcus aureus protein that captures antibodies via specific binding to its Fc domain, has advantages over other chromatographic techniques, such as its high-recovery capacity, selectivity, and robustness, which is preferred for the purification of antibodies of different origins as long as the bond is strong, as is the case with rabbit IgG (Huse et al, 2002).

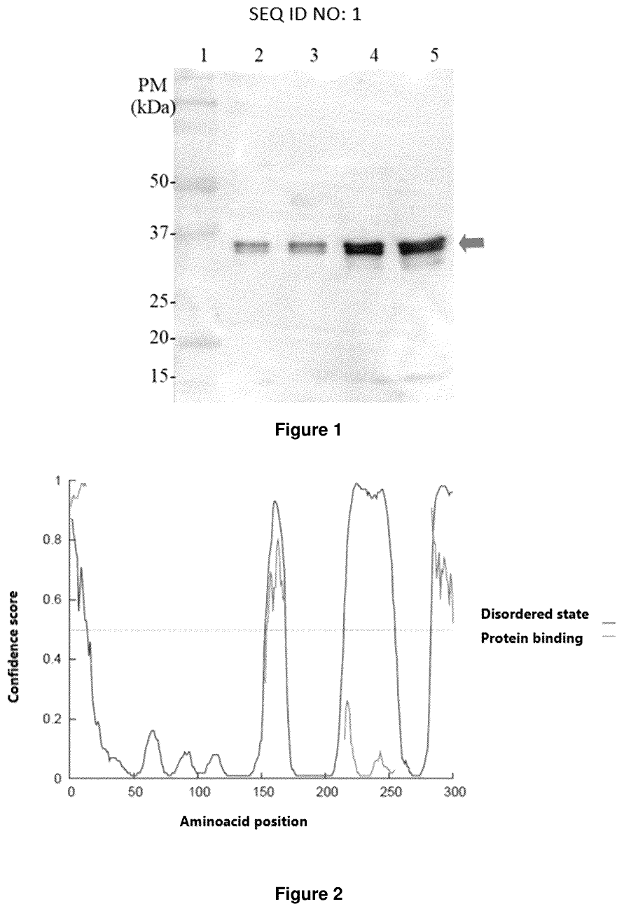

The purified polyclonal antibodies from rabbits 56 and 57 were able to detect up to 20 ng of SEQ ID NO. 1 at a dilution of 1:10,000 (FIG. 1). Considering this dilution and a 10 mL volume of the primary antibody solution for a 96-well plate ELISA assay or a WB membrane, the volume of the anti-SEQ ID NO. 1 polyclonal antibodies.

The anti-SEQ ID NO: 1 polyclonal antibodies were used to corroborate the identity of the peptides SEQ ID NO: 1, SEQ ID NO: 3, and SEQ ID NO:4, as shown in FIG. 1.

### EXAMPLES

### Example 1. Design and Production of an Anti-SEQ NO: 1 Polyclonal Antibody

The nucleotide sequence was optimized to obtain a synthetic gene that encodes the protein SEQ ID NO: 1; the cloning system was perfected to obtain excellent yields and a purity>90%. SEQ ID NO:1 was used to design and produce the anti-SEQ ID NO: 1 polyclonal antibodies, which were later used for peptide identification.

The anti-SEQ ID NO:1 polyclonal antibodies present in the rabbit serum had titers in the order of 1.07×10⁴. In relation to sensitivity, the purified anti-SEQ ID NO:1 polyclonal antibodies from the rabbit sera were able to detect up to 20 ng of SEQ ID NO:1 using a 1:10,000 dilution. The volume obtained from the purified antibodies was sufficient to perform up to 2,225,000 ELISA or Western blot assays.

The antibodies in the sera were purified by affinity chromatography with and without fractional precipitation, both with a purity greater than 90%.

Fractional precipitation of the serum proteins with 35% SAS and affinity chromatography allowed 67% of IgG to be recovered with a purity greater than 96%, while direct serum processing in the affinity chromatography recovered 97.7% of IgG with a purity of 96.8%.

The anti-SEQ ID NO. 1 polyclonal antibodies were purified from the sera of rabbits 56 and 57, with yields and purities greater than 90%. These antibodies detected 20 ng of the protein of SEQ ID NO. 1, with a dilution of 1:10,000.

The anti-SEQ ID NO: 1 polyclonal antibodies were used to corroborate the identity of the peptides SEQ ID NO: 1, SEQ ID NO: 3, and SEQ ID NO:4, as shown in FIG. 1.

### Example 2. Obtaining Peptides Derived from SEQ ID NO: 1 by Enzymatic Proteolysis

Several peptides of various sizes that derive from SEQ ID NO:1 were obtained by enzymatic proteolysis. Two serine proteases were used, trypsin and Lys-C, with weights of 23.5 and 33 kDa, respectively; both selectively cut at the carboxyl end of the lysine residues, and in the case of trypsin, at the arginine residues. Tables 1 and 2 show the peptides derived from SEQ ID NO:1 that were generated by the trypsin and Lys-C proteolysis, respectively.

TABLE 1
Peptides of SEQ ID NO: 1
generated from trypsin proteolysis.

| | | Mass |
|---|---|---|
| Position | Sequence | (Da) |
| 1-6 | MIVNSK | 691.8 |
| 8-12 | SELDK | 591.6 |
| 14-19 | ISIAAK | 602.7 |
| 23-33 | SANAAEITPSR | 1117.2 |
| 34-41 | SSNEELEK | 935.9 |
| 42-45 | ELNR | 531.5 |
| 49-79 | AVGSLETA YKPFLASSALVPTTPTAFQNELK | 3253.71 |
| 83-90 | DSLISSCK | 852.9 |
| 93-126 | NILITDTSSW LGFQVYSTQAPSVQAASTLGF | 3675.1 |
| | ELK | |
| 127-134 | AINSLVNK | 859.0 |
| 135-142 | LAECGLSK | 820.9 |
| 146-184 | VYRPQL PIETPANNPEESDE | 4424.8 |
| 185-189 | ESVLK | 575.6 |
| 190-208 | AMNAITGMQDYLFTVNSIR | 2146.4 |
| 214-263 | MMPPPIANPAAAKPAAAQPATGAASLTPADEA | 4837.6 |
| | AAPAAPAQQVIKPYMGK | |
| 264-281 | EQVFVQVSLNLVHFNQPK | 2127.4 |
| 282-292 | AQEPSEDIEGR | 1231.2 |
| 293-300 | HHHHHHHDI | 1070.1 |

TABLE 2
Peptides of SEQ ID No: 1
generated from proteolysis with Lys-C.

| | | Mass |
|---|---|---|
| Position | Sequence | (Da) |
| 1-22 | MIVNSKRSELDKKISIAAKEIK | 2502.0 |
| 23-41 | SANAAEITPSRSSNEELEK | 2034.1 |
| 42-48 | ELNRYAK | 894.01 |
| 49-58 | AVGSLETAYK | 1039.1 |
| 59-79 | PFLASSALVPTTPTAFQNELK | 2233.5 |
| 80-90 | TFRDSLISSCK | 1257.4 |
| 93-126 | NILITDTSSWLGFQVYSTQAPSVQAASTLG | 3675.1 |
| | FELK | |
| 127-134 | AINSLVNK | 859.0 |
| 135-142 | LAECGLSK | 820.9 |
| 146-189 | VYRPQLPIETPANNPEESDEADQAPWTPMPL | 4981.5 |
| | EIAFQGDRESVLK | |
| 190-226 | AMNAITGMQDYLFTVNSIRIRNERMMPPPIA | 4105.8 |
| | NPAAAK | |
| 227-258 | PAAAQPATGAASLTPADEAAAPAAPAIQQVI | 2970.3 |
| | K | |
| 264-281 | EQVFVQVSLNLVHFNQPK | 2127.4 |
| 282-300 | AQEPSEDIEGRHHHHHHDI | 2282.3 |

### Example 3. Obtaining and Characterizing the Peptides SEQ ID NO: 3 and SEQ ID NO: 4

Derived from the bioinformatic analysis, an optimization of the nucleotide sequences based on the preferential use of E. coli codons was carried out using the OptimumGene program with the aim of obtaining, among others, the peptides SEQ ID NO: 3 and SEQ ID NO: 4, which resulted in the sequences SEQ ID NO: 6 and SEQ ID NO: 7, respectively.

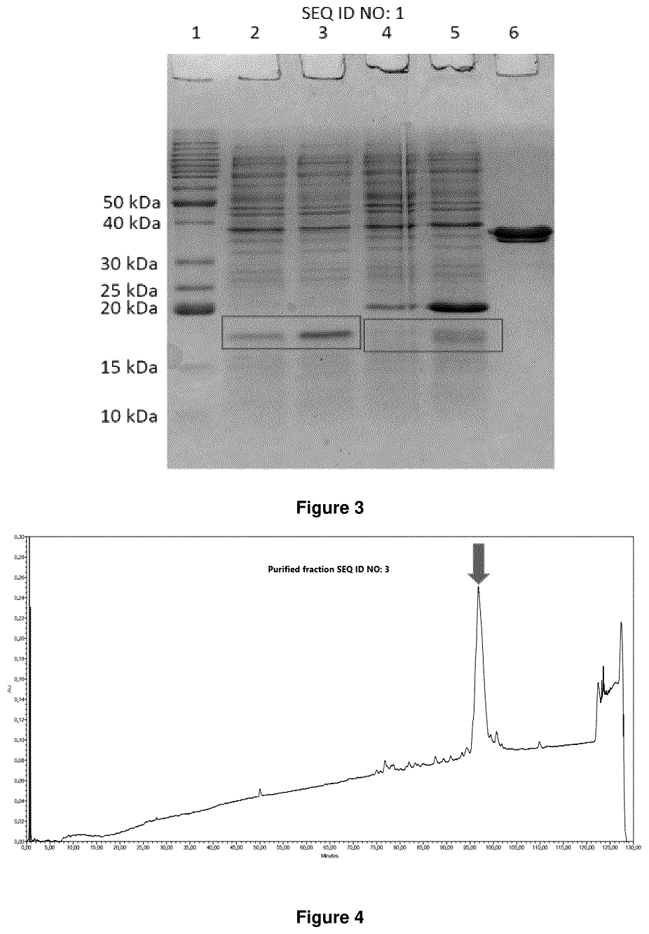

The cF1 and cF2 constructs were designed for the expression of the peptides SEQ ID NO: 4 and SEQ ID NO:3, respectively; they were inspired by a vector/host system pET26b (+)/BL21 and based on the promoter and DNA polymerase of bacteriophage T7. The cloning vectors were generated from the digestion of the plasmid SEQ ID NO: 1_CONTROL pET26b (+) (plasmid designed to obtain SEQ ID No. 1) with the enzymes Ndel and Xhol. The expected fragment sizes for cF1 were 5.2 and 0.47 bp, and for cF2 they were 5.2 and 0.44 kb. Once cF1 and cF2 were obtained and sequenced, the transformation for each was carried out in BL21. Confirmation of the expression of the peptides SEQ ID NO: 4 and SEQ ID NO:3 was performed by agarose gel electrophoresis (FIG. 3) and Western blot. For the Western blot, the anti-SEQ ID NO: 1 antibody was used, which was able to recognize SEQ ID NO: 1, SEQ ID NO: 3, and SEQ ID NO: 4 (FIG. 1).

### Example 4. Obtaining the Peptides SEQ ID NO: 2, SEQ ID NO: 8, SEQ ID NO:9, SEQ ID NO: 10, SEQ ID NO:11, SEQ ID NO:12. SEQ ID NO:13, SEQ ID NO:14, SEQ ID NO:15, SEQ ID NO: 16

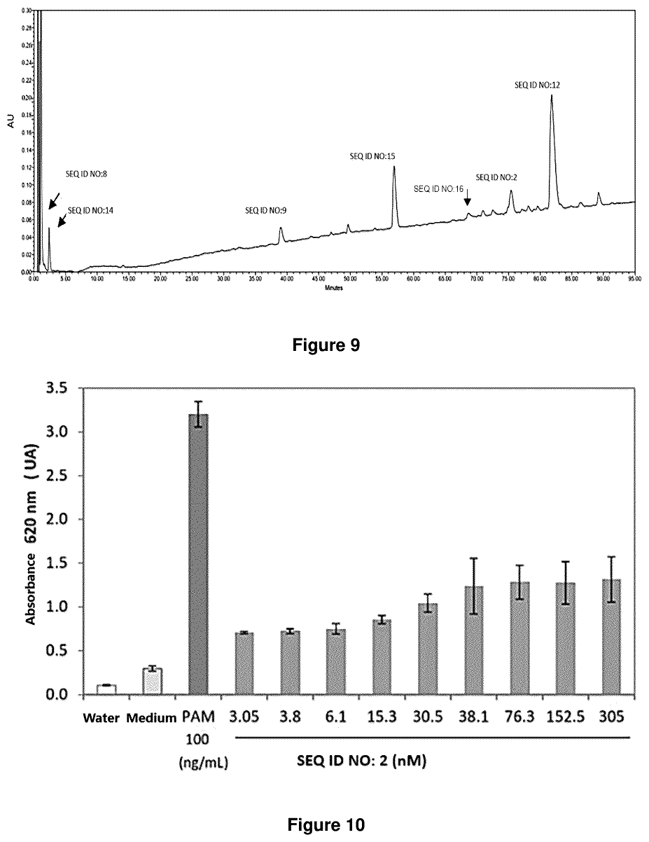

The peptides SEQ ID NO:2, SEQ ID NO:8, SEQ ID NO:9, SEQ ID NO:10, SEQ ID NO: 11, SEQ ID NO: 12, SEQ ID NO: 13, SEQ ID NO: 14, SEQ ID NO:15, SEQ ID NO:16 were generated by proteolysis of the carboxyl terminal of SEQ ID NO:4 with the enzymes trypsin (FIG. 8) and Lys-C (FIG. 9), with each digestion carried out for 18 h at 37° C. The proteolysate was analyzed by UPLC coupled with a mass detector, the identity of the peptides (Tables 3 and 4) was attributed based on the peptides predicted by the bioinformatic analysis and the ionization patterns. The peptides SEQ ID NO: 8, SEQ ID NO: 9, and SEQ ID NO: 12 were obtained with both trypsin and Lys-C. The proteolysis with both proteases was practically complete.

The separation of the proteolysate was carried out by cation exchange, according to their isoelectric point, and by ultrafiltration using a membrane with a cutoff of 5 and 10 kDa.

### Example 5. In Vitro Activity Testing

The in vitro biological activity of SEQ ID NO:2, SEQ ID NO:3, SEQ ID NO:4, SEQ ID NO: 8, SEQ ID NO:9, SEQ ID NO: 10, SEQ ID NO:11, SEQ ID NO: 12, SEQ ID NO: 13, SEQ ID NO: 14, SEQ ID NO: 15, and SEQ ID NO:16 was tested via the stimulation of hTLR2 in the cultures of HEK-Blue hTLR2 cells in 96-well plates for 24 h. After collecting the supernatant, the plate was incubated with QUANTI-Blue at 37° C. for 1 h, and absorbance was measured at 620 nm.

To determine the EC50, the absorbance was plotted against the logarithm of protein concentration, and the data was adjusted to a dose-response sigmoidal ligand binding model with 4 parameters. The cell culture medium was used as a negative control, and PAM (synthetic triacylated lipopeptide (Pam3CSK4)) was used as a positive control.

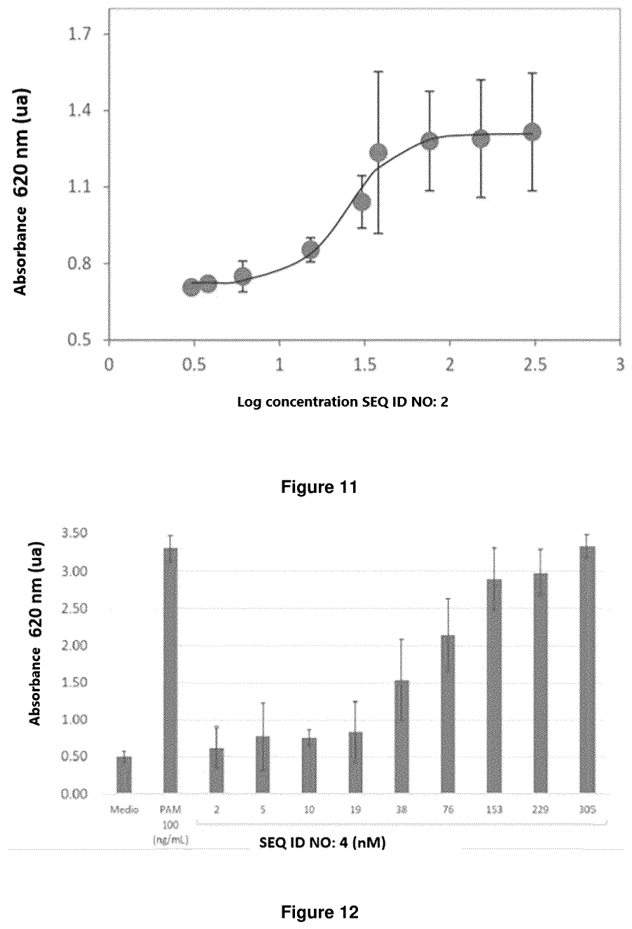

The results for SEQ ID NO: 2 are shown in FIGS. 10 and 11. The results show that SEQ ID NO: 2 stimulates starting at 3.05 nM and obtains the best result at 305 nM.

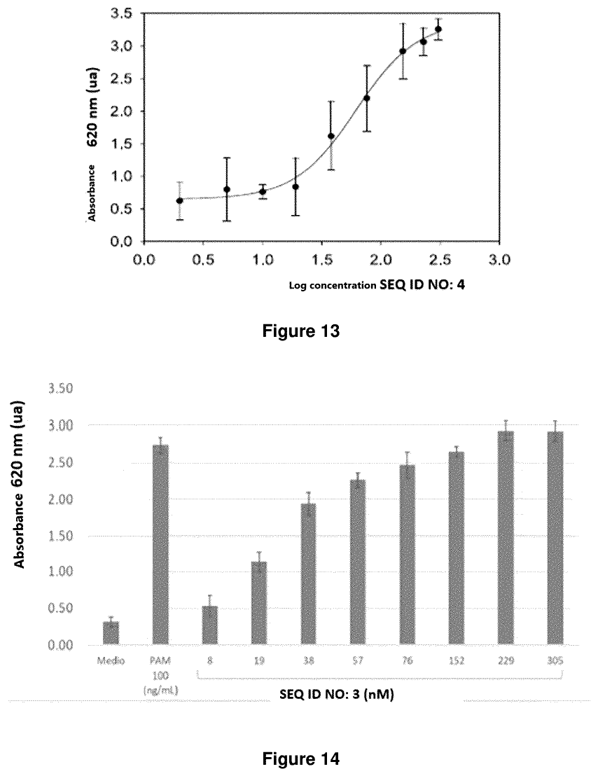

The results for SEQ ID NO: 3 are shown in FIGS. 12 and 13. It's clear from the graphs that SEQ ID NO: 3 significantly stimulated hTLR2 starting at 8 nM. From 229 nM, SEQ ID NO: 3 had a response similar to that of PAM, with a relative response of 107%.

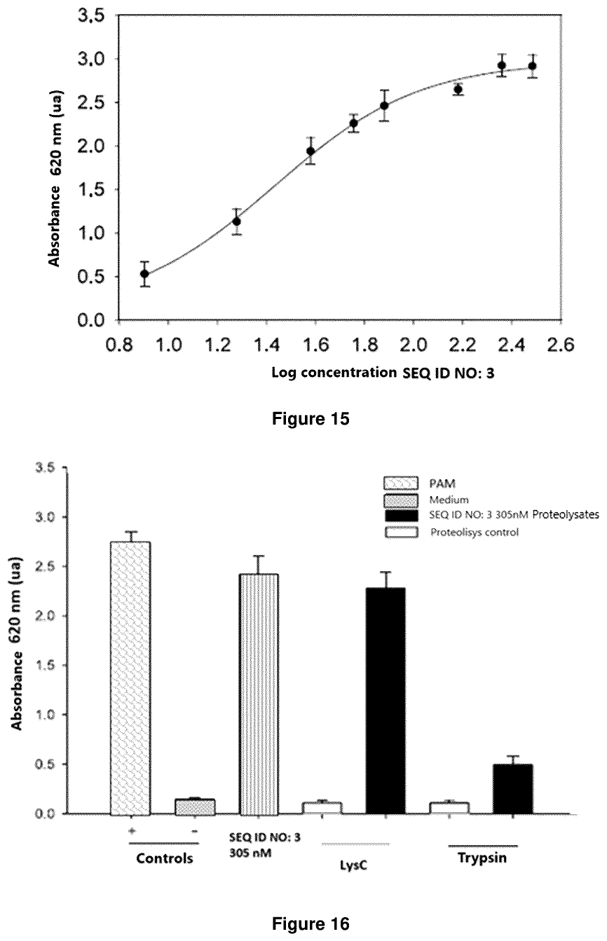

The results for SEQ ID NO: 4 are shown in FIGS. 14 and 15. FIG. 14 shows that SEQ ID NO: 4 significantly stimulated hTLR2 starting at 38 nM. At the maximum concentration tested (305 nM), SEQ ID NO: 4 had a relative response of 101% compared to PAM. The EC50 values of SEQ ID NO: 1, SEQ ID NO: 2, SEQ ID NO: 3, and SEQ ID NO: 4 are shown in Table 3.

TABLE 3
EC₅₀ obtained by stimulation of hTLR2.

| Protein | EC₅₀ (nM) |
|---|---|
| SEQ ID NO: 1 | 35.1 ± 6.4 |
| SEQ ID NO: 2 | 24.9 ± 4.9 |
| SEQ ID NO: 3 | 25.9 ± 5.5 |
| SEQ ID NO: 4 | 65.9.1 ± 22.4 |

FIG. 16 shows that the peptides proteolyzed with Lys-C stimulated a greater response of hTLR2 than the peptides proteolyzed with trypsin, meaning that there are some peptides capable of activating hTLR2 that remain intact after proteolysis with Lys-C, but not with trypsin.

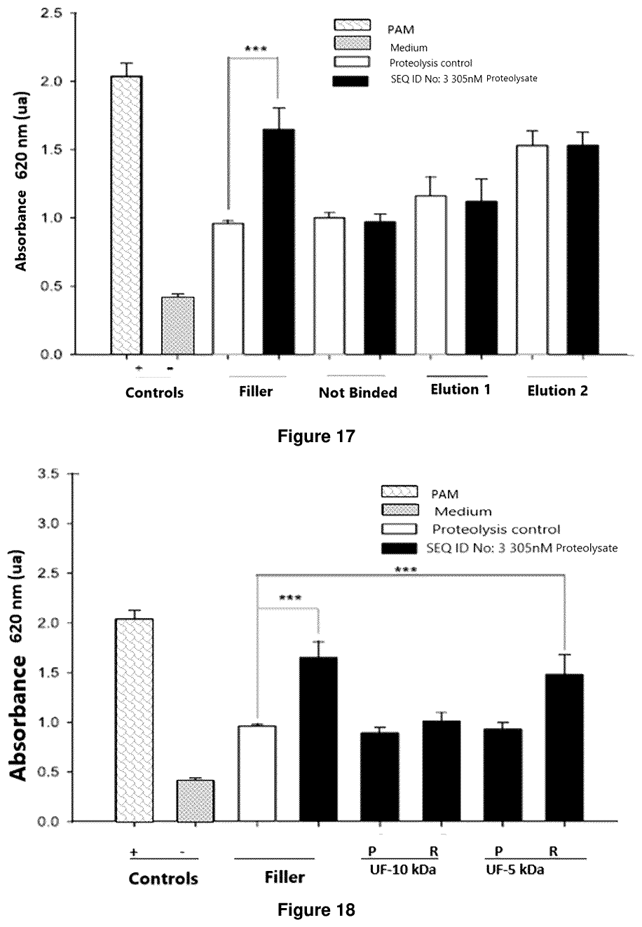

The Lys-C proteolysate was separated by a cation exchanger with a pH greater than 7, using a pH gradient elution. Two elutions were collected: the first (Elution 1) at pH 9, and the second (Elution 2) at pH 10.6. An unbound fraction was also collected up to 2 min of the run. The biological activity of SEQ ID NO: 3, the proteolysis control, and the chromatography fractions are shown in FIG. 17.

In other words, the peptides proteolyzed with Lys-C were separated by ultrafiltration with membranes with 5 and 10 kDa cutoffs. At the 5 kDa cutoff, all the peptides of the proteolysate remained in the retainer with the exception of the peptide SEQ ID NO: 14. At the 10 kDa cutoff, all the peptides with the exception of SEQ ID NO: 16 remained in the permeate. The results of the biological activity assay of the peptides proteolyzed with Lys-C, the proteolysis control, and the respective permeates and retainers of the membranes with 5 and 10 kDa are shown in FIG. 18.

From the above results it can be observed that not all of peptides of the proteolysate have biological activity, and that it is not evident which of the protein fragments are functional.

### Example 6. In Vivo Activity Testing

The effect of the peptides SEQ ID NO: 2 and SEQ ID NO: 3 on the metabolic alterations in mice fed a high-fat diet was evaluated with activity tests in vivo. The in vivo tests included: a glucose tolerance test, an insulin resistance test, a lipid profile, an inflammatory profile and process, and cytokines produced in adipose tissue.

For all the in vivo testing, male mice of the C57BL/6JN strain between 8 to 10 weeks of age were used, housed in groups of 3 to 5 mice per cage with food and water ad libitum in ventilated racks, and whose microbiota were unified for 4 weeks.

Administration of the peptides SEQ ID NO: 2 and SEQ ID NO: 3.

The mice received either 200 μL of a phosphate buffer solution (PBS) at pH 7.5 (as a vehicle), or 200 μL of the PBS solution, with each of the intragastric treatments daily for 12 weeks. They were fed either a Normal Diet (DN, Harlan, Teklad 2018SX) with 18% of the calories coming from fat, or a High-Fat Diet (DAG, Research Diets, D12491), with 60% of the calories coming from fat.

All the mice were randomly divided into five experimental groups in order to study the effect of SEQ ID NO: 3 and SEQ ID NO: 2 correctly:
Group 1—Mice with DN that received 200 μl of PBS intragastrically.
Group 2—Mice with DAG that received 200 μl of PBS intragastrically.
Group 3—Mice with DAG that received 200 μl of PBS with SEQ ID NO: 3 intragastrically at a dose of: 2.5 μg/mouse.
Group 4—Mice with DAG that received 200 μl of PBS with SEQ ID NO: 2 intragastrically at a dose of 0.6 μg/mouse (high dose).
Group 5—Mice with DAG that received 200 μl of PBS with SEQ ID NO: 2 intragastrically at a dose of 0.2 μg/mouse (low dose).

The animal weights and feed intakes were recorded weekly. At week 0, i.e. the initial state, all of the groups showed a similar weight. Table 4 shows that at 12 weeks the mice fed with DAG, regardless of the peptide administration, had an average higher weight compared to those fed with DN.

For the purposes of the present invention, the terms high dose and low dose should be interpreted in accordance with Table 4.

TABLE 4
Dose of peptides SEQ ID No. 3 and SEQ ID NO. 2.

| | | Peptide |
|---|---|---|
| | PM | Administration |
| Peptide | (kDa) | μg/mouse |
| SEQ ID No. 3 | 16.14 | 2.5 |
| SEQ ID NO. 2 | 4.1 | 0.6 |
| High dose | | |
| SEQ ID NO. 2 | 4.1 | 0.2 |
| Low dose | | |

The assay was conducted over 12 weeks, and the results of the tests at 0 weeks and 12 weeks are reported.

To determine the metabolic status of the animals before starting the experiment, weight, basal glucose levels, glucose tolerance, and insulin resistance were recorded. The average weight of the mice on the different diets was measured at 0 weeks and 12 weeks; the results are shown in Table 5.

TABLE 5
Variation in the weight (grams) of the mice
with the different diets at 0 and 12 weeks.

| | | | | DAG+ | DAG+ |
|---|---|---|---|---|---|
| | | | DAG+ | SEQ ID | SEQ ID |
| | | | SEQ ID | NO: 2 | NO: 2 |
| | DN | DAG | NO: 3 | High dose | Low dose |
| 12 weeks | | | | | |
| Average | 28.5 | 43.7 | 37.9 | 42 | 40.2 |
| (grams) | | | | | |
| Standard | 8% | 7% | 12% | 11% | 12% |
| Error | | | | | |
| 0 weeks | | | | | |
| Average | 24.2 | 23.5 | 21.4 | 23.9 | 23.8 |
| (grams) | | | | | |
| Standard | 7% | 8% | 8% | 8% | 8% |
| Error | | | | | |
| n | 6 | 9 | 9 | 9 | 9 |

DN: normal diet;
DAG: high-fat diet;
n: number of mice

### Glucose Tolerance Test (GTT)

The mice were subjected to fasting for 6 hours, and the baseline glucose values in the blood were determined using glucose test strips and a glucometer (Roche's ACCU-CHECK ACTIVE).

Next, the mice received an intraperitoneal injection of 1.8 mg of glucose per gram of body weight, and the blood glucose values were recorded at 15, 30, 60, and 120 minutes after administration.

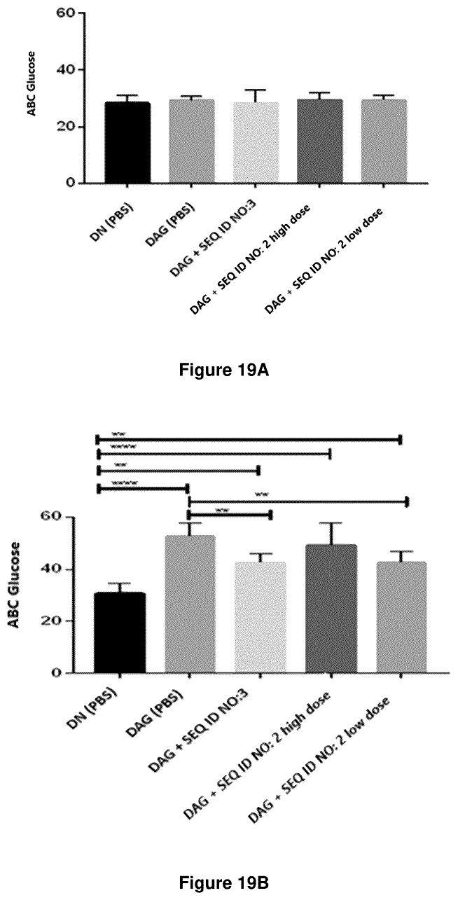

FIGS. 19A and 19B show the area under the curve (AUC) of the glucose tolerance test of the mice, measured at week 0 and 12.

At week 0, no significant variation was observed between the five groups of mice. However, at 12 weeks it is clear that the SEQ ID NO: 2 and SEQ ID NO: 3 groups were statistically different from the DAG group, meaning that SEQ ID NO: 2 and SEQ ID NO:3 improved glucose tolerance.

### Insulin Resistance Test (PRI)

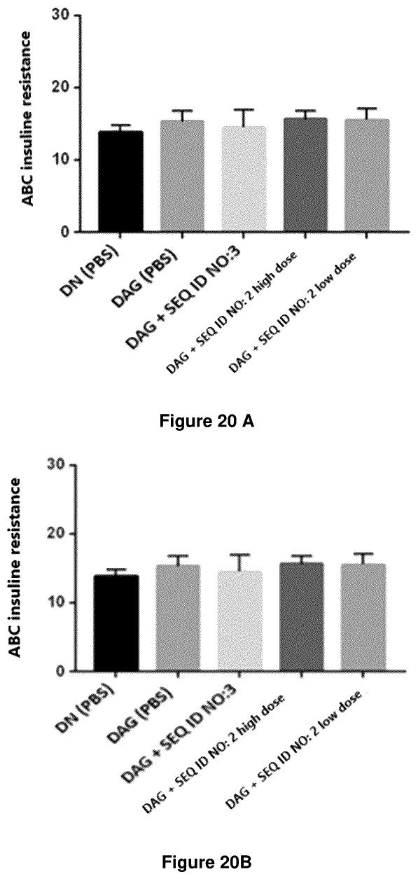

The mice were subjected to fasting for 6 hours and the baseline glucose values were determined, as described above. Subsequently, the mice received an intraperitoneal injection of 1 mU of insulin per gram of body weight, and blood glucose values were recorded at 15, 30, 60, and 120 minutes after administration. The area under the curve (AUC) of the insulin resistance test was calculated considering the glucose decrease between time T0 and T15 using the absolute glucose values, as shown in FIGS. 20A and 20B. The peptides SEQ ID NO: 2 and SEQ ID NO:3 improve insulin sensitivity at 12 weeks of trial compared to the DAG group.

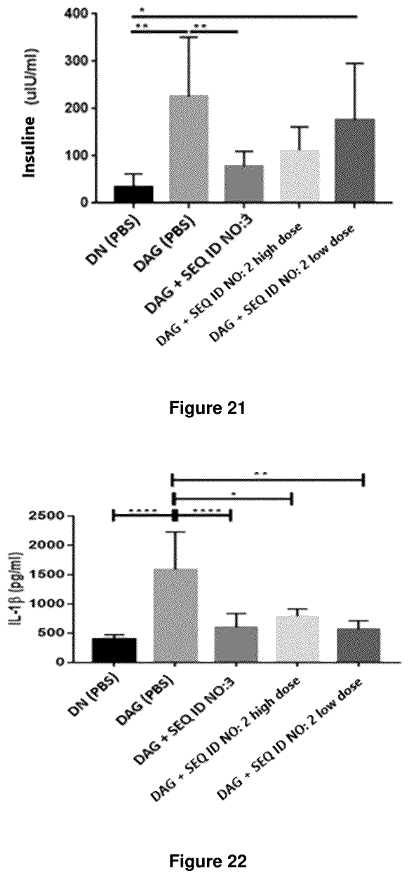

FIG. 21 shows insulin secretion in response to glucose administration, the peptide SEQ ID NO:3 showed a statistically significant reduction in insulin secretion, while the peptide SEQ ID NO:2 shows a tendency to decrease insulin secretion. This means that the peptide SEQ ID NO: 3 improves insulin sensitivity, preventing insulin resistance from developing.

### Inflammatory Profile and Process

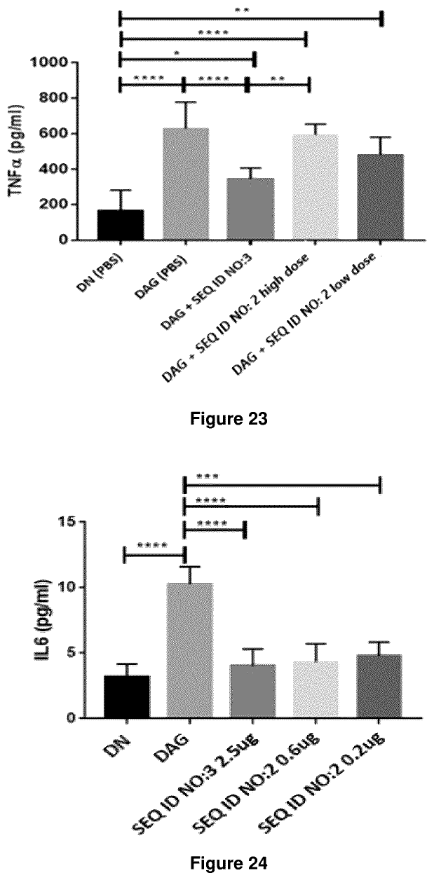

To determine the effect of the peptides SEQ ID NO: 2 and SEQ ID NO: 3 in attenuating the inflammatory process in adipose tissue, the cellular infiltrate of the adipose tissue of the mice was quantified after they had been fed DAG. As a result, the adipose tissue of the animals fed the DAG had a higher cellular infiltrate than the adipose tissue of the animals fed the DN. Notably, the administration of peptides SEQ ID NO: 2 and SEQ ID NO: 3 prevented the increase in cellular infiltrate induced by DAG. In addition, the administration of the peptides SEQ ID NO: 2 and SEQ ID NO: 3 also prevented the increase in the circulating levels of IL-1B (FIG. 22) and TNFα (FIG. 23) induced by DAG, including SEQ ID NO: 2 at low doses. Overall, this data indicates that the peptides SEQ ID NO:3 and SEQ ID NO:2, in high or low doses, attenuate the inflammatory process in adipose tissue resulting from excess energy.

With respect to IL-6, the administration of the peptides SEQ ID NO: 2 and SEQ ID NO: 3 resulted in a dramatic decrease in IL-6 levels for animals fed DAG, practically reaching the same levels as those presented by animals fed DN. FIG. 24 shows the levels of IL-6, in which the groups of animals that were administered the peptides SEQ ID NO: 2 and SEQ ID NO: 3 showed levels practically equal to the levels of the DN-fed mice.

### Hormones Produced in Adipose Tissue.

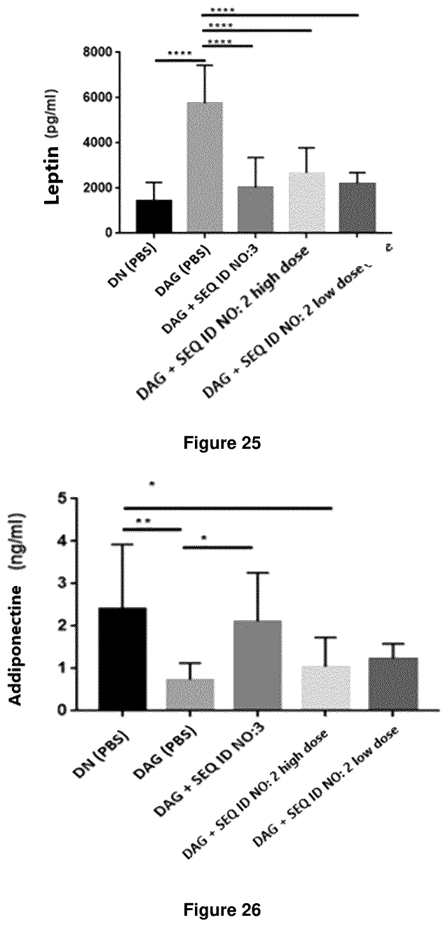

Surprisingly, a decrease in leptin secretion was observed in mice fed SEQ ID NO: 2 and SEQ ID NO: 3 compared to mice fed only DAG (FIG. 25), i.e., the administration of any of the peptides of the present invention results in the control and prevention of increased leptin levels as compared to the levels reported in animals that were fed DAG. On the other hand, it was found that the administration of the peptides SEQ ID NO: 2 and SEQ ID NO: 3 shows a tendency to prevent a fall of adiponectin in circulation induced by DAG (FIG. 26). Both adiponectin and leptin are cytokines secreted by adipose tissue, which have a pleiotropic effect on the physiology of metabolic, inflammatory, and cardiovascular diseases.

Overall, this data shows that the administration of any of the peptides in the invention prevents the metabolic, inflammatory, and endocrine imbalance caused by a DAG diet.

### Example 7. Physicochemical Parameters and Solubility

An in silico prediction of the physicochemical parameters of SEQ ID NO: 4, SEQ ID NO: 3 and the complete protein of SEQ ID NO:1 was performed. The computer tools ProtParam and Protein-Sol were used, as shown in Table 6.

TABLE 6
Estimated physicochemical parameters and solubility of SEQ
ID NO. 1, SEQ ID No. 4, SEQ ID NO. 3, and SEQ ID NO. 2.

| | SEQ ID | SEQ ID | SEQ ID | SEQ ID |
|---|---|---|---|---|
| Parameter | NO: 1 | NO: 4 | NO: 3 | NO: 2 |
| Isoelectric point | 6.40 | 9.34 | 5.08 | 9.98 |
| (pl) | | | | |
| ε_{280 nm} (M⁻¹cm⁻¹) | 19940 | 11460 | 8480 | 1490 |
| Instability Index | 47.98 (un- | 40.77 (un- | 47.17 (un- | 39.87 |
| (II) | stable) | stable) | stable) | |
| Aliphatic Index | 81.47 | 87.71 | 72.09 | 76.76 |
| GRAVY | −0.354 | −0.326 | −0.465 | −0.135 |
| Solubility | 0.528 | 0.680 | 0.816 | 0.65 |

The above results show that SEQ ID NO: 4, SEQ ID NO: 3, and SEQ ID NO: 2 have better solubility and generally better stability than SEQ ID NO: 1.

Experts in the state of the art will recognize, or be able to arrive at, many equivalents of the specified modalities of this invention using no more than routine experimentation.

This invention is not intended to be limited to the modalities and examples provided herein only for illustrative purposes.

1. A characterized peptide because it consists of an amino acid sequence that has at least 80% sequence identity with an amino acid sequence selected from the group consisting of SEQ ID NO: 2, SEQ ID NO: 3, and SEQ ID NO: 4.

2. The peptide in accordance with claim 1, characterized because the sequence identity is at least 90% with SEQ ID NO. 1.

3. The peptide in accordance with either of the claims 1 to 2, characterized because it is a peptide variant of SEQ ID NO: 2, SEQ ID NO: 3, or SEQ ID NO: 4 that has 1, 2, 3, 4, 5, 6, 7, 8, 9 or 10 substituted, removed, and/or added amino acids.

4. The peptide in accordance with either of claims 1 to 2, characterized because it has a length of between 30 and 45 amino acids, or a length of between 112 and 168 amino acids, or a length of between 120 and 180 amino acids.

5. The peptide in accordance with claim 1, characterized because it consists of a sequence selected from SEQ ID NO: 2, SEQ ID NO: 3, and SEQ ID NO: 4.

6. A characterized peptide because it comprises an amino acid sequence that has at least 80% sequence identity with any of the sequences established in SEQ ID NO: 2, SEQ ID NO: 3, or SEQ ID NO: 4, in which the peptide has no more than 280 amino acids in length.

7. A nucleic acid molecule encoding a peptide in accordance with any of the claims 1 to 6.

8. An expression vector comprising a nucleic acid molecule in accordance with claim 7, in which the nucleic acid molecule is operationally bound to a promoter.

9. A host cell comprising an expression vector in accordance with claim 8.

10. A characterized pharmaceutical composition because it comprises a peptide in accordance with any of the claims 1 to 6.

11. The pharmaceutical composition of claim 10, characterized because it comprises pharmaceutically acceptable excipients.

12. The peptide of claims 1 to 6, or the pharmaceutical composition of claims 10-11, for use in the prevention and treatment of inflammatory, metabolic, and endocrine disorders, hyperglycemia, insulinemia, hypercholesterolemia, hypertension, to modulate immune signaling, intestinal barrier function, to maintain the homeostasis of glucose, insulin, adiponectin, leptin, interleukin, cholesterol, and/or triglycerides.

13. The peptide of claims 1 to 6, or the pharmaceutical composition of claims 10-11, which are adapted for oral, parenteral, topical, sublingual, respiratory, or transdermal administration, or any combination thereof.

14. An optimized nucleotide sequence encoding the protein SEQ ID NO: 1, characterized because it comprises SEQ ID NO. 17.

15. The peptide of SEQ ID NO. 1, characterized because it is obtained from the nucleotide sequence SEQ ID NO. 17

16. A method for obtaining a peptide consisting of an amino acid sequence that has at least 80% sequence identity with an amino acid sequence selected from the group consisting of SEQ ID NO: 2, SEQ ID NO: 3, and SEQ ID NO: 4, characterized because the method involves culturing a host cell so that the peptide is expressed under conditions that induce peptide expression, and optionally, separate, extract, and/or purify the cultured peptide;
where the host cell comprises an expression vector that comprises a nucleic acid molecule that encodes the aforementioned peptide.

17. A method to obtain a peptide in accordance with claim 15, in which the peptide is secreted by the cell.

18. A method to obtain a peptide in accordance with claim 15 via the proteolysis of a protein or a purified, bacterial lysate.

19. An anti-SEQ ID NO. 1, SEQ ID NO. 2, SEQ ID NO. 3 and SEQ ID No. 4. polyclonal antibody.


SEQUENCE LISTING (17 sequences)

SEQ ID NO: 1 - protein, 300 aa, synthetic construct
MIVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSEDIEGRHHHHHHDI

SEQ ID NO: 2 - protein, 37 aa, synthetic construct
AMNAITGMQDYLFTVNSIRIRNERMMPPPIANPAAAK

SEQ ID NO: 3 - protein, 139 aa, synthetic construct
QLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSED

SEQ ID NO: 4 - protein, 149 aa, synthetic construct
IVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQ

SEQ ID NO: 5 - DNA, 111 bp, synthetic construct
  source 1..111: mol_type = other DNA
gcgatgaacgcgattaccggcatgcaagattacctgtttaccgttaacagcatccgtattcgtaacgagcgtatgatgccgccgccaatcgcgaacccggcggcggcgaag

SEQ ID NO: 6 - DNA, 417 bp, synthetic construct
  source 1..417: mol_type = other DNA
cagctgccgattgaaaccccggcgaacaacccggaagaaagcgatgaagcggatcaggcgccgtggaccccgatgccgctggaaattgcgtttcagggcgatcgcgaaagcgtgctgaaagcgatgaacgcgattaccggcatgcaggattatctgtttaccgtgaacagcattcgcattcgcaacgaacgcatgatgccgccgccgattgcgaacccggcggcggcgaaaccggcggcggcgcagccggcgaccggcgcggcgagcctgaccccggcggatgaagcggcggcgccggcggcgccggcgattcagcaggtgattaaaccgtatatgggcaaagaacaggtgtttgtgcaggtgagcctgaacctggtgcattttaaccagccgaaagcgcaggaaccgagcgaagat

SEQ ID NO: 7 - DNA, 447 bp, synthetic construct
  source 1..447: mol_type = other DNA
atcgtgaatagcaaacgtagcgaactggataagaaaatcagcattgcggcgaaagaaatcaaaagcgcgaacgcggcggaaattaccccgagccgtagcagcaacgaggaactggagaaagaactgaaccgttacgcgaaggcggtgggtagcctggaaaccgcgtataaaccgttcctggcgagcagcgcgctggttccgaccaccccgaccgcgttccaaaacgaactgaagacctttcgtgacagcctgatcagcagctgcaagaaaaagaacatcctgattaccgataccagcagctggctgggcttccaggtgtacagcacccaagcgccgagcgttcaggcggcgagcaccctgggttttgagctgaaagcgattaacagcctggtgaacaagctggcggaatgcggcctgagcaaatttatcaaggtttatcgtccgcag

SEQ ID NO: 8 - protein, 7 aa, synthetic construct
AQEPSED

SEQ ID NO: 9 - protein, 53 aa, synthetic construct
NERMMPPPIANPAAAKPAAAQPATGAASLTPADEAAAPAAPAIQQVIKPYMGK

SEQ ID NO: 10 - protein, 50 aa, synthetic construct
MMPPPIANPAAAKPAAAQPATGAASLTPADEAAAPAAPAIQQVIKPYMGK

SEQ ID NO: 11 - protein, 19 aa, synthetic construct
AMNAITGMQDYLFTVNSIR

SEQ ID NO: 12 - protein, 41 aa, synthetic construct
MQLPIETPANNPEESDEADQAPWTPMPLEIAFQGDRESVLK

SEQ ID NO: 13 - protein, 36 aa, synthetic construct
MQLPIETPANNPEESDEADQAPWTPMPLEIAFQGDR

SEQ ID NO: 14 - protein, 5 aa, synthetic construct
PYMGK

SEQ ID NO: 15 - protein, 32 aa, synthetic construct
PAAAQPATGAASLTPADEAAAPAAPAIQQVIK

SEQ ID NO: 16 - protein, 18 aa, synthetic construct
EQVFVQVSLNLVHFNQPK

SEQ ID NO: 17 - DNA, 861 bp, synthetic construct
  source 1..861: mol_type = other DNA
atcgtgaatagcaaacgtagcgaactggataagaaaatcagcattgcggcgaaagaaatcaaaagcgcgaacgcggcggaaattaccccgagccgtagcagcaacgaggaactggagaaagaactgaaccgttacgcgaaggcggtgggtagcctggaaaccgcgtataaaccgttcctggcgagcagcgcgctggttccgaccaccccgaccgcgttccaaaacgaactgaagacctttcgtgacagcctgatcagcagctgcaagaaaaagaacatcctgattaccgataccagcagctggctgggcttccaggtgtacagcacccaagcgccgagcgttcaggcggcgagcaccctgggttttgagctgaaagcgattaacagcctggtgaacaagctggcggaatgcggcctgagcaaatttatcaaggtttatcgtccgcagctgccgattgaaaccccggcgaacaacccggaggaaagcgacgaagcggatcaggcgccgtggaccccgatgccgctggagatcgcgttccagggtgaccgtgaaagcgtgctgaaagcgatgaacgcgattaccggcatgcaagattacctgtttaccgttaacagcatccgtattcgtaacgagcgtatgatgccgccgccaatcgcgaacccggcggcggcgaagccggcggcggcgcagccggcgaccggtgcggcgagcctgaccccggcggatgaggcggcggcgccggcggcgccggcgatccagcaagtgattaaaccgtatatgggcaaggaacaagtgttcgttcaagtgagcctgaatctggtgcattttaatcaaccgaaagcgcaagaaccgagcgaagac